(19) United States
(12) Patent Application Publication
(10) Pub. No.: US 2026/0320791 A1
(43) Pub. Date: Oct. 15, 2026

(54) AI-ASSISTED ENDOSCOPIC ULTRASOUND SYSTEM AND METHOD FOR PANCREATIC ABNORMALITY DETECTION

(71) Applicant: Amrita Vishwa Vidyapeetham, Kochi (IN)

(72) Inventors: Gilad GRESSEL, Kollam (IN); Priya NAIR, Kochi (IN); Abijith RAMESH, Kollam (IN); Krishnan GAUTAM P, Kollam (IN); Anshuman SWAIN, Kollam (IN)

(21) Appl. No.: 19/642,787

(22) Filed: Apr. 9, 2026

(30) Foreign Application Priority Data
Apr. 9, 2025 (IN) 202541034844

(51) Int. Cl. G06T 7/00; A61B 8/00; A61B 8/08; A61B 8/12; G06V 10/764; G06V 10/82

(52) U.S. Cl. CPC G06T 7/0012; A61B 8/085; A61B 8/12; A61B 8/42; A61B 8/463; A61B 8/488; A61B 8/5223; G06V 10/764; G06V 10/82; G06T 2200/24; G06T 2207/10016; G06T 2207/10068; G06T 2207/10132; G06T 2207/20081; G06T 2207/20084; G06T 2207/30096; G06V 2201/031

(57) ABSTRACT

The present disclosure relates to an AI-assisted endoscopic ultrasound (EUS) system and a method for real-time analysis of a live endoscopic ultrasound (EUS) video feed. The system includes an EUS device, one or more user interfaces, and a computing unit including one or more processors, a memory, and a hardware accelerator. The computing unit executes an ensemble of deep learning models including a convolutional neural network model, a transformer model, or a combination thereof to identify pancreatic stations, detect pancreatic abnormalities, segment a pancreas and one or more tumours, and optionally extract features from Doppler images. An uncertainty score is calculated and filtered using a calibrated threshold to determine high-confidence predictions. The high-confidence predictions are superimposed on the live EUS video feed, and real-time annotation updates the ensemble in a continuous learning framework.

### TECHNICAL FIELD

The present disclosure generally relates to the field of medical imaging and computer-assisted diagnosis. In particular, the present disclosure relates to an AI-assisted endoscopic ultrasound (EUS) system and method for real-time analysis of a live endoscopic ultrasound (EUS) video feed to support pancreatic station identification, pancreas and tumour segmentation, and detection of pancreatic abnormalities including pancreatic cancer.

### BACKGROUND

Endoscopic ultrasound procedures are widely used for imaging internal anatomical structures and are particularly relevant for evaluating pancreatic conditions including screening and assessment for pancreatic cancer. During an endoscopic ultrasound procedure, an endoscopist typically interprets a live endoscopic ultrasound video feed in real time to recognize anatomical stations, assess tissue appearance, and decide whether a region exhibits an abnormality that warrants further clinical action such as biopsy. Because the live endoscopic ultrasound video feed may vary across patients and may present complex visual patterns, accurate interpretation often depends heavily on operator experience and sustained attention throughout the procedure.

In practice, identification of pancreatic stations and detection of pancreatic abnormalities, including pancreatic cancer, from a live endoscopic ultrasound video feed may be challenging due to variability in imaging perspectives, differences in tissue characteristics, and the need to make time-sensitive judgments during real-time navigation. Such challenges may contribute to variability in procedural assessments and may increase the likelihood of missed findings, including missed cancerous findings, or inconsistent characterization of pancreatic abnormalities.

Although machine learning techniques have been explored for medical image analysis, deploying an AI-assisted workflow during a live endoscopic ultrasound procedure raises additional challenges. Real-time analysis may require efficient processing of successive frames while maintaining reliable outputs suitable for presentation. Further, training robust deep learning models for endoscopic ultrasound may be constrained by limited availability of large, high-quality labeled datasets, and practical workflows for generating real-time annotations may be limited in conventional systems, thereby limiting continuous improvement of model performance over time.

Furthermore, no integrated platform exists that combines station identification, pancreas and tumour segmentation, and cancer detection in a single real-time framework. Existing systems typically address these tasks in isolation, requiring separate workflows and introducing inefficiencies in clinical practice. An integrated approach would streamline the diagnostic workflow, reduce manual intervention, and improve diagnostic consistency by simultaneously processing and presenting these critical outputs within a unified visual interface. Therefore, there is a need for an improved approach that overcomes at least the above-mentioned drawbacks.

### OBJECTS OF THE PRESENT DISCLOSURE

An object of the present disclosure is to provide an AI-assisted endoscopic ultrasound (EUS) system and method for real-time analysis of a live endoscopic ultrasound video feed to support detection of pancreatic abnormalities including pancreatic cancer.

Another object of the present disclosure is to provide automated classification of one or more frames of the live endoscopic ultrasound video feed to identify one or more pancreatic stations and to detect pancreatic abnormalities including pancreatic cancer.

Another object of the present disclosure is to provide segmentation of a pancreas and one or more tumours from one or more frames of the live endoscopic ultrasound video feed to support visualization and assessment during an endoscopic ultrasound procedure.

Another object of the present disclosure is to provide an integrated platform combining station identification, segmentation, and cancer detection that streamlines clinical workflows and improves diagnostic consistency.

Another object of the present disclosure is to provide an uncertainty estimation and filtering approach in which an uncertainty score is calculated for one or more predictions and predictions are filtered using a calibrated uncertainty threshold to determine one or more high-confidence predictions for real-time presentation.

Another object of the present disclosure is to provide real-time superimposition of the one or more high-confidence predictions on the live endoscopic ultrasound video feed for assisting an endoscopist during the endoscopic ultrasound procedure.

Another object of the present disclosure is to provide real-time annotation of the live endoscopic ultrasound video feed and to use the real-time annotation in a continuous learning framework to improve performance of one or more deep learning models over time.

Another object of the present disclosure is to provide an AI-assisted endoscopic ultrasound workflow that reduces variability in pancreatic station identification and abnormality detection including pancreatic cancer detection, thereby improving reliability of intra-procedure assessment.

### SUMMARY

Aspects of the present disclosure pertain to the field of medical imaging and computer-assisted diagnosis. In particular, the present disclosure relates to an AI-assisted endoscopic ultrasound (EUS) system and method for real-time analysis of a live endoscopic ultrasound (EUS) video feed.

An aspect of the present disclosure pertains to an AI-assisted endoscopic ultrasound system. The system includes an endoscopic ultrasound device configured to generate a live endoscopic ultrasound video feed. The system further includes one or more user interfaces configured to receive, from an endoscopist, information indicative of a current scope location. The system also includes a computing unit operatively coupled to the endoscopic ultrasound device and the one or more user interfaces. The computing unit is configured to receive the live endoscopic ultrasound video feed from the endoscopic ultrasound device. The computing unit is configured to execute an ensemble of deep learning models including at least one convolutional neural network (CNN) model, at least one transformer model, or a combination thereof to analyze the live endoscopic ultrasound video feed. The computing unit is configured to receive the information indicative of the current scope location via the one or more user interfaces. The computing unit is configured to perform, on one or more frames of the live endoscopic ultrasound video feed, classification to identify one or more pancreatic stations and detect one or more pancreatic abnormalities including at least pancreatic cancer. The computing unit is configured to perform segmentation of a pancreas and one or more tumours within the one or more frames. The computing unit is configured to calculate an uncertainty score for each prediction generated by the ensemble of deep learning models. The computing unit is configured to filter predictions based on a calibrated uncertainty threshold to determine one or more high-confidence predictions. The computing unit is configured to superimpose, in real time, the one or more high-confidence predictions on the live endoscopic ultrasound video feed. The computing unit is also configured to enable real-time annotation of the live endoscopic ultrasound video feed. The real-time annotation is used to update the ensemble of deep learning models in a continuous learning framework. In one embodiment, the computing unit is further configured to perform extraction of one or more features from one or more Doppler images.

In one embodiment, the ensemble of deep learning models includes one or more models selected from the group consisting of ResNet, DenseNet, EfficientNet, other CNNs and Vision Transformers or other transformer based models.

In one embodiment, the one or more user interfaces include one or more interfaces selected from the group including a touchscreen device, a voice command recognition system, and a custom-built hardware device including dedicated buttons.

In one embodiment, the AI-assisted endoscopic ultrasound system further includes a hardware accelerator configured to accelerate processing of the live endoscopic ultrasound video feed.

In one embodiment, the AI-assisted endoscopic ultrasound system further includes a cloud-based server in communication with the computing unit. The cloud-based server is configured to store one or more complete endoscopic ultrasound procedure videos. The cloud-based server is also configured to store the real-time annotation associated with the live endoscopic ultrasound video feed. The cloud-based server stores the one or more complete endoscopic ultrasound procedure videos and the real-time annotation for training and refinement of at least one deep learning model of the ensemble of deep learning models.

In one embodiment, calculating the uncertainty score includes comparing outputs generated by a plurality of deep learning models in the ensemble of deep learning models to quantify prediction consistency.

In one embodiment, the computing unit is configured to generate a biopsy-location guidance output indicating one or more optimal biopsy locations. The biopsy-location guidance output is based on analysis of the segmented tumour regions. The biopsy-location guidance output is also based on one or more surrounding tissue characteristics derived from the live endoscopic ultrasound video feed. The biopsy-location guidance output is further based on one or more blood flow patterns derived from the one or more Doppler images.

In one embodiment, superimposition of the one or more high-confidence predictions on the live endoscopic ultrasound video feed is selectable by the endoscopist to be toggled on or off.

In one embodiment, the AI-assisted endoscopic ultrasound system further includes an auxiliary monitor operatively coupled to the computing unit. The computing unit is configured to display, on the auxiliary monitor, additional AI-generated information. The additional AI-generated information includes segmentation outlines and classification labels corresponding to the live endoscopic ultrasound video feed.

Another aspect of the present disclosure pertains to a method for AI-assisted endoscopic ultrasound. The method includes generating, by an endoscopic ultrasound device, a live endoscopic ultrasound video feed. The method further includes receiving, by a computing unit operatively coupled to the endoscopic ultrasound device, the live endoscopic ultrasound video feed from the endoscopic ultrasound device. The method includes receiving, by one or more user interfaces, from an endoscopist, information indicative of a current scope location. The method further includes receiving, by the computing unit, the information indicative of the current scope location via the one or more user interfaces. The method includes executing, by the computing unit, an ensemble of deep learning models including at least one convolutional neural network model, at least one transformer model, or a combination thereof to analyze the live endoscopic ultrasound video feed. The method includes performing, by the computing unit on one or more frames of the live endoscopic ultrasound video feed, classification to identify one or more pancreatic stations and detect one or more pancreatic abnormalities including at least pancreatic cancer. The method also includes segmentation of a pancreas and one or more tumours within the one or more frames. The method includes calculating, by the computing unit, an uncertainty score for each prediction generated by the ensemble of deep learning models. The method includes filtering predictions based on a calibrated uncertainty threshold to determine one or more high-confidence predictions. The method includes superimposing, by the computing unit in real time, the one or more high-confidence predictions on the live endoscopic ultrasound video feed. The method further includes enabling, by the computing unit, real-time annotation of the live endoscopic ultrasound video feed. The real-time annotation is used to update the ensemble of deep learning models in a continuous learning framework. Additionally, in one embodiment, the method may further include extracting one or more features from one or more Doppler images.

### BRIEF DESCRIPTION OF THE DRAWINGS

The accompanying drawings illustrate exemplary embodiments of the present disclosure and, together with the description, serve to explain the principles of the present disclosure.

FIG. 1 illustrates an example block diagram of an AI-assisted endoscopic ultrasound (EUS) system, in accordance with an embodiment of the present disclosure.

FIG. 2 illustrates an exemplary block diagram of the computing unit, in accordance with an embodiment of the present disclosure.

FIG. 3 illustrates an exemplary block diagram of the cloud-based server, in accordance with an embodiment of the present disclosure.

FIG. 4 illustrates an exemplary flow chart of a method for AI-assisted endoscopic ultrasound (EUS), in accordance with an embodiment of the present disclosure.

### DETAILED DESCRIPTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

The following is a detailed description of embodiments of the disclosure depicted in the accompanying drawings. The embodiments are in such detail as to clearly communicate the disclosure. If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Accurate interpretation of a live endoscopic ultrasound video feed during an endoscopic ultrasound procedure is technically challenging because the endoscopist must identify pancreatic stations and evaluate suspected pancreatic abnormalities in real time under variable imaging perspectives and tissue characteristics. This reliance on manual interpretation may lead to variability in assessment, and it is further constrained by limited availability of high-quality annotated data suitable for training and improving analysis models in a practical workflow.

The disclosed approach provides an AI-assisted endoscopic ultrasound system and method that receive a live endoscopic ultrasound video feed and an input indicative of a current scope location, and analyze frames of the live endoscopic ultrasound video feed using an ensemble of deep learning models including at least one convolutional neural network model, at least one transformer model, or a combination thereof. The system performs classification to identify pancreatic stations and detect pancreatic abnormalities including pancreatic cancer and segmentation of a pancreas and one or more tumours. In one embodiment, the system may further perform extraction of features from Doppler images. The system then calculates an uncertainty score for each prediction and filters predictions using a calibrated uncertainty threshold. High-confidence predictions are superimposed in real time on the live endoscopic ultrasound video feed, and real-time annotation is enabled so that the ensemble of deep learning models may be updated in a continuous learning framework.

The disclosed approach enables real-time, uncertainty-filtered presentation of model outputs on the live endoscopic ultrasound video feed, thereby improving reliability of displayed predictions and supporting more consistent identification of pancreatic stations and pancreatic abnormalities during the procedure. Segmentation and Doppler-derived features provide additional structured information for assessment, while real-time annotation supports continuous learning updates that may progressively refine model performance using procedure-derived data.

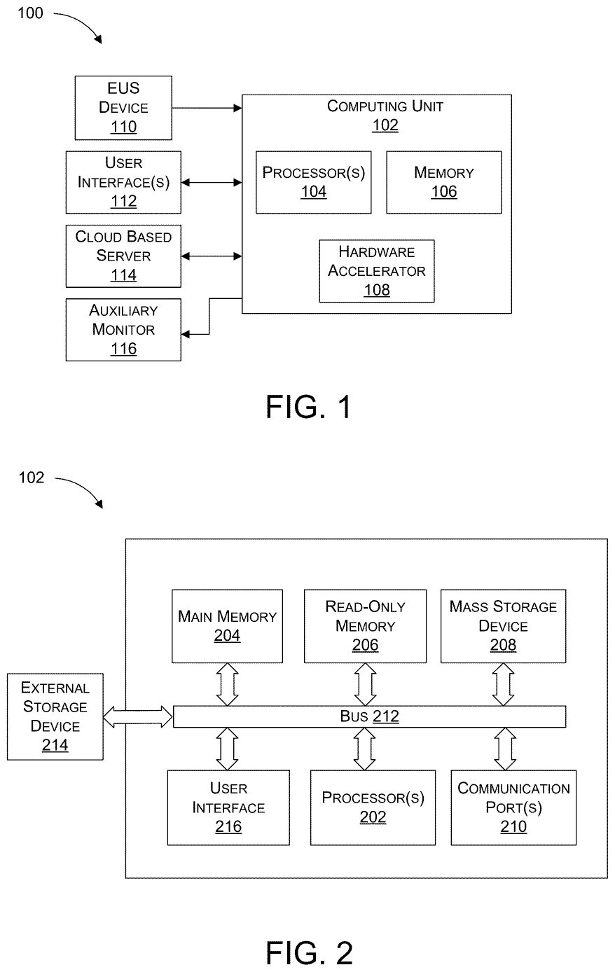

Various embodiments with respect to the present disclosure will be explained in detail with reference to FIGS. 1-4.

FIG. 1 illustrates an example block diagram of an AI-assisted endoscopic ultrasound (EUS) system 100, in accordance with one or more embodiments of the present disclosure.

The AI-assisted endoscopic ultrasound (EUS) system 100 may include an EUS device 110 and a computing unit 102 operatively coupled with the EUS device 110. The AI-assisted endoscopic ultrasound (EUS) system 100 may further include one or more user interfaces 112 operatively coupled with the computing unit 102, a cloud-based server 114 operatively coupled with the computing unit 102, and an auxiliary monitor 116 operatively coupled with the computing unit 102.

The EUS device 110 may be configured to generate a live endoscopic ultrasound (EUS) video feed and to provide the live endoscopic ultrasound (EUS) video feed to the computing unit 102. In one embodiment, the EUS device 110 may include an endoscopic ultrasound imaging apparatus that outputs a stream of ultrasound frames for real-time processing, but not limited thereto.

The one or more user interfaces 112 may be configured to receive, from an endoscopist, information indicative of a current scope location. The one or more user interfaces 112 may provide the information indicative of the current scope location to the computing unit 102. In one embodiment, the one or more user interfaces 112 may include a touchscreen interface, a voice command recognition interface, or a custom-built hardware interface including dedicated buttons, but not limited thereto.

The computing unit 102 may include one or more processor(s) 104, a memory 106, and a hardware accelerator 108. The one or more processor(s) 104 may be operatively coupled with the memory 106 and the hardware accelerator 108. The memory 106 may store instructions that, when executed by the one or more processor(s) 104, cause the computing unit 102 to process the live endoscopic ultrasound (EUS) video feed received from the EUS device 110 and to generate outputs for presentation. The hardware accelerator 108 may be configured to accelerate at least a portion of processing of the live endoscopic ultrasound (EUS) video feed, including executing one or more deep learning inference operations, but not limited thereto.

The cloud-based server 114 may be configured to exchange data with the computing unit 102. In one embodiment, the cloud-based server 114 may store one or more EUS procedure videos and associated annotations received from the computing unit 102 for subsequent training and refinement of one or more deep learning models, but not limited thereto.

The auxiliary monitor 116 may be configured to receive display data from the computing unit 102. In one embodiment, the auxiliary monitor 116 may present additional AI-generated information associated with the live endoscopic ultrasound (EUS) video feed, including one or more segmentation outlines and one or more classification labels, but not limited thereto.

FIG. 2 illustrates an exemplary block diagram of the computing unit 102, in accordance with one or more embodiments of the present disclosure. The computing unit 102 may include one or more processors 202 (may also be referred to as one or more processor(s) 104), a main memory 204 (may also be referred to as memory 106), a read-only memory 206, a mass storage device 208, one or more communication port(s) 210, a bus 212, an external storage device 214, and a user interface 216. The elements of the computing unit 102 may be operatively coupled to one another via the bus 212.

The one or more processors 202 may include one or more general-purpose processors, microprocessors, microcontrollers, digital signal processors, system-on-chip devices, or application-specific integrated circuits, but not limited thereto. The one or more processors 202 may be configured to fetch and execute instructions stored in one or more memory components of the computing unit 102. The one or more processors 202 may thereby support real-time processing of a live endoscopic ultrasound (EUS) video feed and generation of analysis outputs, as described in connection with the AI-assisted endoscopic ultrasound (EUS) system 100.

The main memory 204 may include volatile memory that may be used to temporarily store program instructions, runtime data, and intermediate results during execution of inference and analysis operations. In one embodiment, the main memory 204 may store one or more frame buffers corresponding to the live endoscopic ultrasound (EUS) video feed, intermediate feature maps generated by deep learning models, and uncertainty values computed for model predictions, but not limited thereto. The read-only memory 206 may include non-volatile memory that may store boot firmware, initialization routines, and one or more configuration parameters used to initialize operation of the computing unit 102.

The mass storage device 208 may include non-transitory storage that may store software modules, configuration files, logged output data, and one or more trained model parameters associated with the ensemble of deep learning models, but not limited thereto. The external storage device 214 may be operatively coupled to the bus 212 and may provide removable or attachable storage for importing or exporting data. In one embodiment, the external storage device 214 may include removable media or a portable storage device for transferring procedure records, software updates, or model updates, but not limited thereto.

The one or more communication port(s) 210 may be configured to enable data communication between the computing unit 102 and one or more external components. In one embodiment, the one or more communication port(s) 210 may enable reception of the live endoscopic ultrasound (EUS) video feed from the EUS device 110 and exchange of data with the cloud-based server 114 and the auxiliary monitor 116 shown in FIG. 1, but not limited thereto.

The user interface 216 may be configured to enable local interaction with the computing unit 102. In one embodiment, the user interface 216 may include one or more input/output components that allow an operator or an endoscopist to provide information indicative of a current scope location and to view analysis outputs generated from the live endoscopic ultrasound (EUS) video feed, but not limited thereto.

The bus 212 may include one or more interconnects configured to transfer data and control signals among the one or more processors 202, the main memory 204, the read-only memory 206, the mass storage device 208, the one or more communication port(s) 210, the external storage device 214, and the user interface 216. The configuration shown in FIG. 2 is illustrative. One or more components of the computing unit 102 may be consolidated, distributed, or implemented using a different arrangement, without departing from the scope of the present disclosure.

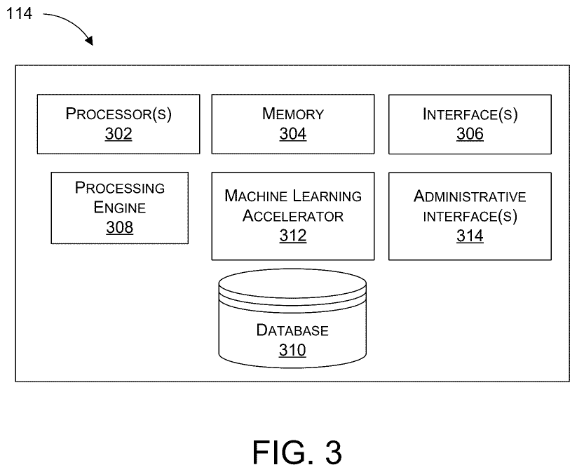

FIG. 3 illustrates an exemplary block diagram of the cloud-based server 114, in accordance with one or more embodiments of the present disclosure. The cloud-based server 114 may provide centralized, distributed, or cloud-hosted computational resources for supporting one or more operations associated with the AI-assisted endoscopic ultrasound (EUS) system 100. In one embodiment, the cloud-based server 114 may be operatively coupled with the computing unit 102 of FIG. 1 to receive data for storage and to support model training or model refinement workflows, but not limited thereto.

The cloud-based server 114 may include one or more processor(s) 302 configured to execute computer-readable instructions associated with server-side processing. In one embodiment, the one or more processor(s) 302 may be configured to coordinate storage of one or more complete EUS procedure videos and associated annotations received from the computing unit 102, but not limited thereto.

The cloud-based server 114 may include a memory 304 that may provide volatile and non-volatile storage for program instructions, configuration parameters, and runtime data. In one embodiment, the memory 304 may store one or more training configurations, uncertainty threshold parameters, or model version information associated with an ensemble of deep learning models, but not limited thereto.

The cloud-based server 114 may include one or more interface(s) 306 configured to enable bidirectional communication with external systems. In one embodiment, the one or more interface(s) 306 may enable data exchange with the computing unit 102 over one or more wired or wireless networks to receive EUS procedure videos, receive annotations, and provide one or more updated model artifacts or configuration data, but not limited thereto.

The cloud-based server 114 may include a processing engine 308 configured to coordinate one or more server-side workflows. In one embodiment, the processing engine 308 may manage ingestion of uploaded data, scheduling of training jobs, validation of model updates, and packaging of refined model parameters for deployment to the computing unit 102, but not limited thereto.

The cloud-based server 114 may include a database 310 configured to store persistent data associated with operation of the AI-assisted endoscopic ultrasound (EUS) system 100. In one embodiment, the database 310 may store one or more complete EUS procedure videos, real-time annotations, derived metadata, and one or more trained model parameters corresponding to the ensemble of deep learning models, but not limited thereto.

The cloud-based server 114 may include a machine learning accelerator 312 configured to accelerate execution of one or more machine-learning operations. In one embodiment, the machine learning accelerator 312 may accelerate training, fine-tuning, or batch inference for refining one or more deep learning models based on stored EUS procedure videos and associated annotations, but not limited thereto.

The cloud-based server 114 may include one or more administrative interface(s) 314 configured to enable administrative operations. In one embodiment, the one or more administrative interface(s) 314 may enable authorized personnel to manage data retention, initiate or schedule model update cycles, review model versions, or configure access permissions for stored EUS procedure videos and annotations, but not limited thereto.

The configuration shown in FIG. 3 is illustrative. One or more functional components of the cloud-based server 114 may be consolidated, distributed across multiple servers, virtualized, or deployed in a cloud computing environment without departing from the scope of the present disclosure.

In one embodiment, an AI-assisted endoscopic ultrasound (EUS) system 100 may include an EUS device 110 configured to generate a live endoscopic ultrasound (EUS) video feed. The AI-assisted endoscopic ultrasound (EUS) system 100 may further include one or more user interfaces 112 configured to receive, from an endoscopist, information indicative of a current scope location. The AI-assisted endoscopic ultrasound (EUS) system 100 may also include a computing unit 102 operatively coupled to the EUS device 110 and the one or more user interfaces 112.

In one embodiment, the computing unit 102 may be configured to receive the live endoscopic ultrasound (EUS) video feed from the EUS device 110. The computing unit 102 may be configured to execute an ensemble of deep learning models to analyze the live endoscopic ultrasound (EUS) video feed. In one embodiment, the ensemble of deep learning models may include at least one convolutional neural network (CNN) model, at least one transformer model, or a combination thereof, but not limited thereto.

In one embodiment, the computing unit 102 may be configured to receive the information indicative of the current scope location via the one or more user interfaces 112. The computing unit 102 may be configured to process one or more frames of the live endoscopic ultrasound (EUS) video feed based on the information indicative of the current scope location.

In one embodiment, the computing unit 102 may be configured to perform, on the one or more frames of the live endoscopic ultrasound (EUS) video feed, classification to identify one or more pancreatic stations and detect one or more pancreatic abnormalities including pancreatic cancer. The computing unit 102 may be configured to perform segmentation of a pancreas and one or more tumours within the one or more frames. The computing unit 102 may be configured to perform extraction of one or more features from one or more Doppler images.

In one embodiment, the computing unit 102 may be configured to calculate an uncertainty score for each prediction generated by the ensemble of deep learning models. The computing unit 102 may be configured to filter predictions based on a calibrated uncertainty threshold to determine one or more high-confidence predictions.

In one embodiment, the computing unit 102 may be configured to execute the ensemble of deep learning models such that the ensemble of deep learning models includes one or more models selected from the group consisting of ResNet, DenseNet, EfficientNet, other CNNs and Vision Transformers or other transformer based models. In one embodiment, at least one model of the ensemble of deep learning models may be a ResNet model, a DenseNet model, an EfficientNet model or other CNN model configured to perform feature extraction and classification on one or more frames of the live endoscopic ultrasound (EUS) video feed, but not limited thereto. In one embodiment, at least one model of the ensemble of deep learning models may be a Transformer based model configured to analyze one or more frames of the live endoscopic ultrasound (EUS) video feed using attention-based representations to support at least one of pancreatic station identification, pancreatic abnormality detection, or segmentation of a pancreas and one or more tumours, but not limited thereto.

In one embodiment, the one or more user interfaces 112 may include one or more interfaces selected from the group including a touchscreen device, a voice command recognition system, and a custom-built hardware device including dedicated buttons. The touchscreen device may be configured to receive the information indicative of the current scope location via one or more touch-based selections, but not limited thereto. The voice command recognition system may be configured to receive the information indicative of the current scope location via one or more spoken inputs provided by the endoscopist, but not limited thereto. The custom-built hardware device may include one or more dedicated buttons configured to allow the endoscopist to input the information indicative of the current scope location during an EUS procedure, but not limited thereto.

In one embodiment, the AI-assisted endoscopic ultrasound (EUS) system 100 may further include a hardware accelerator 108 configured to accelerate processing of the live endoscopic ultrasound (EUS) video feed. The hardware accelerator 108 may be operatively coupled to the computing unit 102. The hardware accelerator 108 may be configured to accelerate one or more operations performed to analyze the live endoscopic ultrasound (EUS) video feed, including executing one or more deep learning inference operations corresponding to the ensemble of deep learning models, but not limited thereto.

In one embodiment, the AI-assisted endoscopic ultrasound (EUS) system 100 may further include a cloud-based server 114 in communication with the computing unit 102. The cloud-based server 114 may be configured to store one or more complete EUS procedure videos and the real-time annotation associated with the live endoscopic ultrasound (EUS) video feed. The cloud-based server 114 may store the one or more complete EUS procedure videos and the real-time annotation for training and refinement of at least one deep learning model of the ensemble of deep learning models. In one embodiment, the computing unit 102 may transmit at least a portion of the one or more complete EUS procedure videos and the real-time annotation to the cloud-based server 114 via one or more communication port(s) 210, but not limited thereto.

In one embodiment, calculating the uncertainty score may include comparing outputs generated by a plurality of deep learning models in the ensemble of deep learning models to quantify prediction consistency. The plurality of deep learning models may generate respective outputs for at least one of pancreatic station identification, pancreatic abnormality detection, or segmentation of a pancreas and one or more tumours. The computing unit 102 may compare the respective outputs to determine a degree of agreement among the plurality of deep learning models, but not limited thereto. The computing unit 102 may use the degree of agreement as a basis for the uncertainty score such that higher agreement corresponds to lower uncertainty and lower agreement corresponds to higher uncertainty, but not limited thereto.

In one embodiment, the computing unit 102 may be configured to generate a biopsy-location guidance output indicating one or more optimal biopsy locations. The biopsy-location guidance output may be generated based on analysis of the segmented tumour regions, one or more surrounding tissue characteristics derived from the live endoscopic ultrasound (EUS) video feed, and one or more blood flow patterns derived from the one or more Doppler images. In one embodiment, the biopsy-location guidance output may identify one or more locations within, adjacent to, or around a segmented tumour region based on the surrounding tissue characteristics and the blood flow patterns, but not limited thereto.

In one embodiment, superimposition of the one or more high-confidence predictions on the live endoscopic ultrasound (EUS) video feed may be selectable by the endoscopist to be toggled on or off. The one or more user interfaces 112 may be configured to receive an input from the endoscopist indicating whether to enable or disable the superimposition, but not limited thereto. In one embodiment, when the superimposition is toggled off, the computing unit 102 may continue to analyze the live endoscopic ultrasound (EUS) video feed without presenting the one or more high-confidence predictions as an overlay, but not limited thereto.

In one embodiment, the AI-assisted endoscopic ultrasound (EUS) system 100 may further include an auxiliary monitor 116 operatively coupled to the computing unit 102. The computing unit 102 may be configured to display, on the auxiliary monitor 116, additional AI-generated information including segmentation outlines and classification labels corresponding to the live endoscopic ultrasound (EUS) video feed. In one embodiment, the auxiliary monitor 116 may present the additional AI-generated information concurrently with presentation of the live endoscopic ultrasound (EUS) video feed, but not limited thereto.

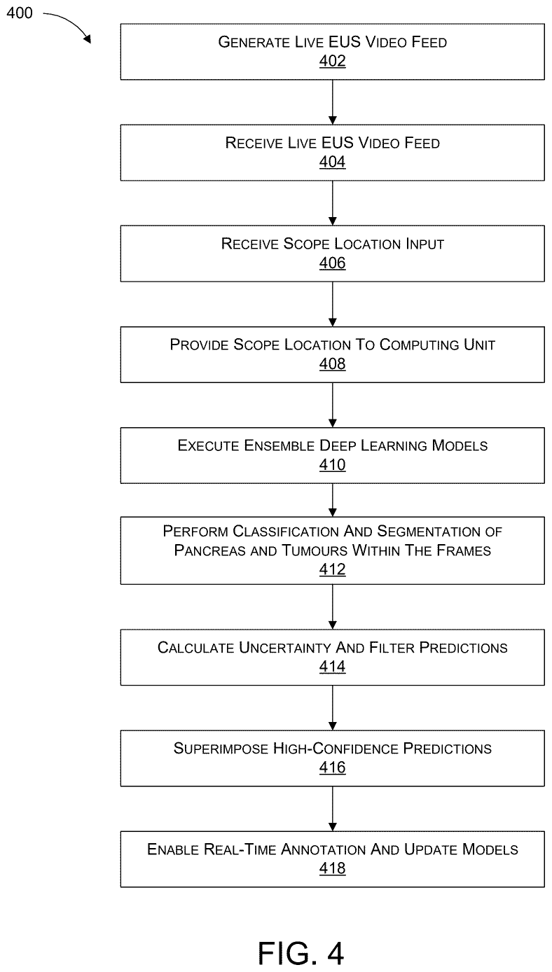

Referring now to FIG. 4, an exemplary flow chart of a method 400 for AI-assisted endoscopic ultrasound (EUS) is illustrated, in accordance with one or more embodiments of the present disclosure. In one embodiment, the method 400 may be implemented by the AI-assisted endoscopic ultrasound (EUS) system 100 that includes the computing unit 102 having the one or more processor(s) 104 and the memory 106. In one embodiment, one or more steps of the method 400 may be accelerated by the hardware accelerator 108.

At step 402, the method 400 may include generating, by the EUS device 110, a live endoscopic ultrasound (EUS) video feed. The live endoscopic ultrasound (EUS) video feed may include a stream of EUS frames acquired during an EUS procedure, but not limited thereto.

At step 404, the method 400 may include receiving, by the computing unit 102 operatively coupled to the EUS device 110, the live endoscopic ultrasound (EUS) video feed from the EUS device 110. In one embodiment, the computing unit 102 may receive the live endoscopic ultrasound (EUS) video feed via the one or more communication port(s) 210, but not limited thereto.

At step 406, the method 400 may include receiving, by the one or more user interfaces 112, from an endoscopist, information indicative of a current scope location. The information indicative of the current scope location may indicate a current station or anatomical region being examined during the EUS procedure, but not limited thereto.

At step 408, the method 400 may include receiving, by the computing unit 102, the information indicative of the current scope location via the one or more user interfaces 112. In one embodiment, the computing unit 102 may associate the information indicative of the current scope location with one or more frames of the live endoscopic ultrasound (EUS) video feed for subsequent analysis, but not limited thereto.

At step 410, the method 400 may include executing, by the computing unit 102, an ensemble of deep learning models to analyze the live endoscopic ultrasound (EUS) video feed. In one embodiment, the ensemble of deep learning models may include at least one convolutional neural network (CNN) model, at least one transformer model, or a combination thereof, but not limited thereto.

At step 412, the method 400 may include performing, by the computing unit 102 on one or more frames of the live endoscopic ultrasound (EUS) video feed, classification to identify one or more pancreatic stations and detect one or more pancreatic abnormalities including pancreatic cancer. The method 400 may further include performing segmentation of a pancreas and one or more tumours within the one or more frames. The method 400 may further include extracting one or more features from one or more Doppler images, but not limited thereto.

At step 414, the method 400 may include calculating, by the computing unit 102, an uncertainty score for each prediction generated by the ensemble of deep learning models. The method 400 may further include filtering predictions based on a calibrated uncertainty threshold to determine one or more high-confidence predictions, but not limited thereto.

At step 416, the method 400 may include superimposing, by the computing unit 102 in real time, the one or more high-confidence predictions on the live endoscopic ultrasound (EUS) video feed. In one embodiment, the superimposing may include overlaying one or more classification labels or one or more segmentation outlines corresponding to the one or more high-confidence predictions, but not limited thereto.

At step 418, the method 400 may include enabling, by the computing unit 102, real-time annotation of the live endoscopic ultrasound (EUS) video feed. The real-time annotation may include one or more user-provided labels, markings, or corrections associated with at least a portion of the live endoscopic ultrasound (EUS) video feed, but not limited thereto. The method 400 may further include using the real-time annotation to update the ensemble of deep learning models in a continuous learning framework.

It may be appreciated that one or more steps of the method 400 may be performed in a different order, combined, omitted, or supplemented with additional steps, without departing from the scope of the present disclosure.

In one embodiment, the AI-assisted endoscopic ultrasound (EUS) system 100 may include a mobile application configured to enable real-time annotation of the live endoscopic ultrasound (EUS) video feed generated by the EUS device 110. The mobile application may be operatively coupled to the computing unit 102 via the one or more communication port(s) 210 to receive, directly or indirectly, at least a portion of the live endoscopic ultrasound (EUS) video feed or one or more frames thereof, but not limited thereto. The mobile application may include a user interface configured to allow an endoscopist or an authorized operator to input one or more annotations corresponding to one or more frames of the live endoscopic ultrasound (EUS) video feed. In one embodiment, the one or more annotations may include one or more labels, markings, corrections, or confirmations associated with at least one of pancreatic station identification, pancreatic abnormality detection, or segmentation of a pancreas and one or more tumours, but not limited thereto.

In one embodiment, the mobile application may be configured to associate the one or more annotations with corresponding frame identifiers, timestamps, or scope-location information received via the one or more user interfaces 112, but not limited thereto. The mobile application may be configured to transmit the one or more annotations to the computing unit 102 for storage in the memory 106 and for use in a continuous learning framework. In one embodiment, the computing unit 102 may be configured to forward at least a portion of the one or more annotations and associated video data to the cloud-based server 114 for subsequent training and refinement of at least one deep learning model of the ensemble of deep learning models, but not limited thereto. The mobile application may further be configured to present, to the endoscopist or the authorized operator, one or more feedback views that include a view of the live endoscopic ultrasound (EUS) video feed with superimposed outputs generated by the computing unit 102, thereby facilitating efficient review and real-time annotation during an EUS procedure, but not limited thereto.

In one embodiment, the computing unit 102 may be configured to use the information indicative of the current scope location received via the one or more user interfaces 112 to contextualize analysis of the live endoscopic ultrasound (EUS) video feed generated by the EUS device 110. In one embodiment, the information indicative of the current scope location may include a depth of scope insertion and may further include gastrointestinal location context indicative of a current anatomical region, such as a stomach region or a duodenum region, but not limited thereto. The computing unit 102 may use the information indicative of the current scope location to support identification of one or more pancreatic stations and to determine whether a pancreas is in view within one or more frames of the live endoscopic ultrasound (EUS) video feed, but not limited thereto.

In one embodiment, the computing unit 102 may be configured to detect one or more pancreatic abnormalities including pancreatic cancer using classification performed on one or more frames of the live endoscopic ultrasound (EUS) video feed. In one embodiment, the one or more pancreatic abnormalities may include pancreatic cancer, chronic calcific pancreatitis, a cyst, or a mass, but not limited thereto. In one embodiment, when segmentation indicates a tumour region within the one or more frames, the computing unit 102 may generate an additional tumour characterization output. The additional tumour characterization output may include an assessment indicative of tumour malignancy, but not limited thereto.

In one embodiment, the computing unit 102 may be configured to calculate the uncertainty score for each prediction generated by the ensemble of deep learning models using one or more uncertainty-related signals. In one embodiment, the one or more uncertainty-related signals may include an internal uncertainty estimate produced by at least one model, an input image quality measure derived from the live endoscopic ultrasound (EUS) video feed, and a consistency measure derived from predictions computed across multiple frames, but not limited thereto. The computing unit 102 may apply a calibrated uncertainty threshold to filter predictions and determine the one or more high-confidence predictions. In one embodiment, the calibrated uncertainty threshold may be specific to a prediction type, including a classification prediction or a segmentation prediction, but not limited thereto. In one embodiment, the computing unit 102 may adjust the calibrated uncertainty threshold based on one or more operational settings or one or more clinical context inputs, but not limited thereto.

In one embodiment, the cloud-based server 114 may be configured to store one or more complete endoscopic ultrasound procedure videos and associated annotation data received from the computing unit 102. The one or more complete endoscopic ultrasound procedure videos may be used for training and refinement of at least one deep learning model of the ensemble of deep learning models. In one embodiment, using the one or more complete endoscopic ultrasound procedure videos may enable the ensemble of deep learning models to learn temporal progression across consecutive frames and to learn from suboptimal views encountered during an endoscopic ultrasound procedure, but not limited thereto. The computing unit 102 may receive one or more updated model parameters from the cloud-based server 114 and may use the one or more updated model parameters for subsequent analysis of the live endoscopic ultrasound (EUS) video feed, but not limited thereto.

In one embodiment, the computing unit 102 may be configured to use real-time annotation received during an endoscopic ultrasound procedure as training data in the continuous learning framework, even when the real-time annotation includes variability across annotators or sessions. The computing unit 102 and the cloud-based server 114 may be configured to apply a noise-tolerant learning approach to reduce the effect of inconsistent or noisy real-time annotation on model refinement, but not limited thereto. In one embodiment, the noise-tolerant learning approach may include weighting, filtering, or selecting portions of the real-time annotation based on agreement across repeated annotations or consistency across multiple frames, but not limited thereto.

In one embodiment, the computing unit 102 may be configured to interface with the EUS device 110 through a video capture interface for acquiring the live endoscopic ultrasound (EUS) video feed. The computing unit 102 may be configured to extract frames from the live endoscopic ultrasound (EUS) video feed and to execute the ensemble of deep learning models using the hardware accelerator 108 to produce predictions with low latency. In one embodiment, the computing unit 102 may be configured to generate and superimpose the one or more high-confidence predictions on the live endoscopic ultrasound (EUS) video feed within a sub-second latency that supports real-time use, but not limited thereto.

In one embodiment, the AI-assisted endoscopic ultrasound (EUS) system 100 may be deployed in different configurations depending on clinical workflow and infrastructure. In one embodiment, at least a portion of the analysis performed by the computing unit 102 may be integrated with the EUS device 110, or at least a portion of the analysis may be performed by a separate computing unit 102 communicatively coupled to the EUS device 110, but not limited thereto. In one embodiment, the superimposed outputs may be presented on a primary display associated with the EUS device 110, and the computing unit 102 may additionally output display data to the auxiliary monitor 116 for presenting additional AI-generated information, but not limited thereto.

In one embodiment, the AI-assisted endoscopic ultrasound (EUS) system 100 may include a mobile application operable on a standard mobile device to support real-time annotation of the live endoscopic ultrasound (EUS) video feed. The mobile application may be configured to present one or more frames of the live endoscopic ultrasound (EUS) video feed and to receive user inputs indicating one or more labels, markings, or corrections as the real-time annotation, but not limited thereto. The mobile application may be configured to transmit the real-time annotation to the computing unit 102 via the one or more communication port(s) 210, and the computing unit 102 may forward the real-time annotation to the cloud-based server 114 for secure storage and subsequent training and refinement of at least one deep learning model, but not limited thereto.

While the foregoing describes example embodiments, other and further embodiments may be devised without departing from the scope defined by the claims, and the embodiments, versions, and examples are non-limiting and are provided solely to enable a person having ordinary skill in the art to make and use the present disclosure in view of their general knowledge.

### ADVANTAGES OF THE PRESENT DISCLOSURE

The present disclosure provides an AI-assisted endoscopic ultrasound system and method that perform real-time analysis of a live endoscopic ultrasound video feed to support pancreatic station identification and pancreatic abnormality detection, including pancreatic cancer detection, during an endoscopic ultrasound procedure.

The present disclosure provides an ensemble of deep learning models including at least one convolutional neural network model and at least one transformer model, thereby improving robustness of inference across variable imaging perspectives and tissue characteristics.

The present disclosure provides segmentation of a pancreas and one or more tumours within frames of the live endoscopic ultrasound video feed, enabling structured visualization that may support clearer interpretation of anatomical boundaries and suspected tumour regions.

The present disclosure provides uncertainty-based filtering by calculating an uncertainty score for predictions and applying a calibrated uncertainty threshold, thereby enabling presentation of high-confidence predictions and reducing unreliable overlays on the live video feed.

The present disclosure provides real-time superimposition of high-confidence predictions on the live endoscopic ultrasound video feed, thereby delivering immediate visual guidance while allowing the endoscopist to maintain a continuous procedural workflow.

The present disclosure provides extraction of one or more features from one or more Doppler images, thereby incorporating blood-flow related information into analysis of pancreatic abnormalities including pancreatic cancer and supporting improved assessment of tumour-related characteristics.

The present disclosure provides real-time annotation of the live endoscopic ultrasound video feed and continuous learning updates to the ensemble of deep learning models, thereby facilitating progressive refinement of performance using procedure-derived data and annotations.

We claim:

1. An AI-assisted endoscopic ultrasound (EUS) system comprising:
an EUS device configured to generate a live endoscopic ultrasound (EUS) video feed;
one or more user interfaces configured to receive, from an endoscopist, information indicative of a current scope location; and
a computing unit operatively coupled to the EUS device and the one or more user interfaces, wherein the computing unit is configured to:
receive the live EUS video feed from the EUS device;
execute an ensemble of deep learning models comprising at least one convolutional neural network (CNN) model, at least one transformer model, or a combination thereof, to analyze the live EUS video feed;
receive the information indicative of the current scope location via the one or more user interfaces;
perform, on one or more frames of the live EUS video feed, classification to identify one or more pancreatic stations and detect one or more pancreatic abnormalities comprising at least pancreatic cancer, and segmentation of a pancreas and one or more tumours within the one or more frames;
calculate an uncertainty score for each prediction generated by the ensemble of deep learning models and filter predictions based on a calibrated uncertainty threshold to determine one or more high-confidence predictions;
superimpose, in real time, the one or more high-confidence predictions on the live EUS video feed; and
enable real-time annotation of the live EUS video feed, wherein the real-time annotation is used to update the ensemble of deep learning models in a continuous learning framework.

2. The AI-assisted EUS system of claim 1, wherein the computing unit is further configured to perform extraction of one or more features from one or more Doppler images.

3. The AI-assisted EUS system of claim 1, wherein the ensemble of deep learning models comprises one or more models selected from the group consisting of ResNet, DenseNet, EfficientNet, other CNNs, and Vision Transformers or other transformer based models.

4. The AI-assisted EUS system of claim 1, wherein the one or more user interfaces comprise one or more interfaces selected from the group comprising a touchscreen device, a voice command recognition system, and a custom-built hardware device including dedicated buttons.

5. The AI-assisted EUS system of claim 1, further comprising a hardware accelerator configured to accelerate processing of the live EUS video feed.

6. The AI-assisted EUS system of claim 1, further comprising a cloud-based server in communication with the computing unit, wherein the cloud-based server is configured to store one or more complete EUS procedure videos and the real-time annotation associated with the live EUS video feed for training and refinement of at least one deep learning model of the ensemble of deep learning models.

7. The AI-assisted EUS system of claim 1, wherein calculating the uncertainty score comprises comparing outputs generated by a plurality of deep learning models in the ensemble of deep learning models to quantify prediction consistency.

8. The AI-assisted EUS system of claim 1, wherein the computing unit is configured to generate a biopsy-location guidance output indicating one or more optimal biopsy locations based on analysis of the segmented tumour regions, one or more surrounding tissue characteristics derived from the live EUS video feed, and one or more blood flow patterns derived from one or more Doppler images.

9. The AI-assisted EUS system of claim 1, wherein superimposition of the one or more high-confidence predictions on the live EUS video feed is selectable by the endoscopist to be toggled on or off.

10. The AI-assisted EUS system of claim 1, further comprising an auxiliary monitor operatively coupled to the computing unit, wherein the computing unit is configured to display, on the auxiliary monitor, additional AI-generated information including segmentation outlines and classification labels corresponding to the live EUS video feed.

11. A method for AI-assisted endoscopic ultrasound (EUS), the method comprising:
generating, by an endoscopic ultrasound (EUS) device, a live endoscopic ultrasound (EUS) video feed;
receiving, by a computing unit operatively coupled to the EUS device, the live EUS video feed from the EUS device;
receiving, by one or more user interfaces, from an endoscopist, information indicative of a current scope location;
receiving, by the computing unit, the information indicative of the current scope location via the one or more user interfaces;
executing, by the computing unit, an ensemble of deep learning models comprising at least one convolutional neural network (CNN) model, at least one transformer model, or a combination thereof, to analyze the live EUS video feed;
performing, by the computing unit on one or more frames of the live EUS video feed, classification to identify one or more pancreatic stations and detect one or more pancreatic abnormalities comprising at least pancreatic cancer and segmentation of a pancreas and one or more tumours within the one or more frames;
calculating, by the computing unit, an uncertainty score for each prediction generated by the ensemble of deep learning models and filtering predictions based on a calibrated uncertainty threshold to determine one or more high-confidence predictions;
superimposing, by the computing unit in real time, the one or more high-confidence predictions on the live EUS video feed; and
enabling, by the computing unit, real-time annotation of the live EUS video feed, wherein the real-time annotation is used to update the ensemble of deep learning models in a continuous learning framework.

12. The method of claim 11, further comprising extracting, by the computing unit, one or more features from one or more Doppler images.
